(19) United States
(12) Patent Application Publication
(10) Pub. No.: US 2010/0249219 A1
(43) Pub. Date: Sep. 30, 2010

(54) SHORT RNA ANTAGONIST COMPOUNDS FOR THE MODULATION OF HIF-1ALPHA

(76) Inventors: Maj HEDTJÄRN, Copenhagen (DK); Jens Bo Rode Hansen, Hellerup (DK)

Correspondence Address: DECHERT LLP, P.O. BOX 390460, MOUNTAIN VIEW, CA, 94039-0460, US

(21) Appl. No.: 12/753,588

(22) Filed: Apr. 2, 2010

Related U.S. Application Data
(63) Continuation-in-part of application No. PCT/EP2008/062658, filed on Sep. 23, 2008.
(60) Provisional application No. 60/977,409, filed on Oct. 4, 2007.

(30) Foreign Application Priority Data
Mar. 19, 2008 (EP) PCT/EP2008/053314

(51) Int. Cl. A61K 31/7088; C07H 21/04; C12N 5/02

(52) U.S. Cl. 514/44 R; 536/23.1; 435/375

(57) ABSTRACT

The present invention relates to oligomeric compounds (oligomers) of 12, 13 or 14 nucleotides in length, which target Hif-1alpha mRNA in a cell, leading to reduced expression of Hif-1alpha. Reduction of Hif-1alpha expression is beneficial for the treatment of certain medical disorders, such as hyperproliferative disorders, such as cancer.

### 1. CROSS REFERENCE TO RELATED APPLICATIONS

This application is a continuation-in-part under 35 U.S.C. §120 of International Patent Application No PCT/EP2008/062658, U.S. Provisional Patent Application Ser. No. 60/977,409, filed on Oct. 4, 2007 and to International Patent Application No. PCT/EP2008/053314, filed on Mar. 19, 2008, and published as WO 2008/113832, each of which is incorporated herein, by reference, in its entirety.

### 2. FIELD OF INVENTION

The present invention relates to short oligomeric compounds (shortmers) that target the Hif-1alpha mRNA in a cell, leading to reduced expression of Hif-1alpha. Reduction of Hif-1alpha expression is beneficial for a range of medical disorders such as hyperproliferative disorders, such as cancer.

### 3. BACKGROUND

LNA antisense oligonuclelotides which target Hif-1alpha are known to be useful for in vivo down-regulation of Hif-1alpha and can be used in therapeutic applications such as the treatment of hyperproliferative disorders such as cancer. WO2006/050734 and WO03/085110 disclose LNA gapmer oligomers which target Hif-1alpha. Specifically, WO2006/050734 discloses LNA gapmer oligomers of formulas 5′-GₓGₓCₛAₛAₛGₛCₛAₛTₛCₛCₛTₓGₓT-3″, 5′-TₓTₓAₛCₛTₛGₛCₛCₛTₛTₛCₛTₓTₓA-3′, 5′-GₛGₛCₛAₛAₛGₛCₛAₛTₛCₛCₛTₛGₛT-3′, or 5′-TₛTₛAₛCₛTₛGₛCₛCₛTₛTₛCₛTₛTₛA-3′ (as disclosed in WO2006/050734) wherein uppercase letters denote a beta-D-oxy-LNA nucleoside analogue, lowercase letters denote a 2′-deoxynucleoside, an underlined letter denotes either a beta-D-oxy-LNA nucleoside analogue or a 2′-deoxynucleoside, subscript “s” denotes a phosphorothioate link between neighbouring nucleosides/LNA nucleoside analogues, and subscript denotes either a phosphorothioate link or a phosphorodiester link between neighbouring nucleosides/LNA nucleoside analogues. There is a need for improved antisense oligonucleotides which target Hif-1alpha.

Citation or identification of any reference in Section 2 or in any other section of this application shall not be construed as an admission that such reference is available as prior art to the present disclosure.

### 4. SUMMARY OF INVENTION

The invention provides an oligomer consisting of 12, 13 or 14 contiguous monomers which has a sequence that is fully complementary to the sequence of a region of SEQ ID NO: 1, wherein said oligomer has a sequence that is identically present in SEQ ID NO: 5, and wherein all internucleoside linkages are phosphorothioate linkages.

The invention provides an oligomer consisting of 12 contiguous monomers which has a sequence that is fully complementary to the sequence of a region of SEQ ID NO 1, wherein said oligomer comprises at least one nucleoside analogue, such as at least one LNA monomer.

The invention provides an oligomer consisting of 12 contiguous monomers which has a sequence that is fully complementary to the sequence of a region of SEQ ID NO: 1, wherein said oligomer consist of the design 5′-A-B-C3′; wherein region A consists of 2 contiguous LNA monomers; region B consists of 8 contiguous DNA monomers, and region C consists of 2 contiguous LNA monomers.

The invention provides an oligomer consisting of a sequence that is identically present in SEQ ID NOs: 20, 21, 22, 23, 24, 25, 26 oar 27.

The invention also provides oligomers having the sequence set forth in SEQ ID NO: 29 and SEQ ID NO: 30.

The invention provides a conjugate comprising the oligomer according to the invention, and at least one non-nucleotide or non-polynucleotide moiety covalently attached to said oligomer.

The invention provides a pharmaceutical composition comprising the oligomer according to the invention, or the conjugate according to the invention, and a pharmaceutically acceptable diluent, carrier, salt or adjuvant.

The invention provides for an oligomer according to the invention, or the conjugate according to the invention, for use as a medicament in the treatment of a medical disorder, such as a hyperproliferative disorder, such as cancer.

The invention provides for the use of the oligomer according to the invention, or a conjugate according to the invention, for the manufacture of a medicament for the treatment of a medical disorder such as a hyperproliferative disorder, such as cancer.

The invention provides for a method of treating a disease or disorder in a subject, such as a hyperproliferative disorder, such as cancer, said method comprising administering to the subject an effective amount of an oligomer according to the invention, or a conjugate according to the invention or a pharmaceutical composition according to the invention.

It should be noted that the indefinite articles “a” and “an” and the definite article “the” are used in the present application, as is common in patent applications, to mean one or more unless the context clearly dictates otherwise. Further, the term “or” is used in the present application, as is common in patent applications, to mean the disjunctive “or” or the conjunctive “and.”

All publications mentioned in this specification are herein incorporated by reference. Any discussion of documents, acts, materials, devices, articles or the like that has been included in this specification is solely for the purpose of providing a context for the present disclosure. It is not to be taken as an admission that any or all of these matters form part of the prior art base or were common general knowledge in the field relevant to the present disclosure as it existed anywhere before the priority date of this application.

The features and advantages of the disclosure will become further apparent from the following detailed description of embodiments thereof.

### 5. BRIEF DESCRIPTION OF FIGURES

FIG. 1: Down-regulation of Hif-1alpha mRNA in mouse liver using a 16mer oligomer (SEQ ID NO: 18), and a series of 12, 13 and 14mer oligomers (see Example 4). NMRI mice were dosed 5 mg/kg/dose on 3 consecutive days (one dose/day i.v.) and animals were sacrificed 24 hours after last dosing. At sacrifice, liver tissue was sampled. RNA was isolated from the tissues and the expression of Hif-1alpha mRNA was measured using qPCR. Reducing the size of the 16mer resulted in a length dependant increase in activity when analyzing Hif-1alpha mRNA down-regulation in liver, with the 12mers being the most potent. The 2-8-2 12mer design was found to be more potent than the 1-9-2 design, and in some embodiments is preferred.

FIG. 2: Down-regulation of Hif-1alpha snRNA in mouse kidney using a 16mer oligomer (SEQ ID NO: 18), and a series of 12, 13 and 14mer oligomers (see Example 4), NMRI mice were dosed 5 mg/kg/dose on 3 consecutive days (one dose/day i.v.) and animals were sacrificed 24 hours after last dosing. At sacrifice, kidney tissue was sampled. RNA was isolated from the tissues and the expression of Hif-1alpha mRNA was measured using qPCR. Reducing the size of the 16mer resulted in a length dependant increase in activity when analyzing Hif-1alpha mRNA down-regulation in kidney, although not as pronounced as those seen in liver with the 2-8-2 12mer being the most potent.

FIG. 3: The amount of oligomer having the design set forth in SEQ ID NO: 29 present in the urine of mouse injected with 1×50 mg/kg at 1 hr, 6 hr, and 24 hrs after injection, and the total amount.

FIG. 4: The amount of oligomer having the design set forth in SEQ ID NO: 30 present in the urine of mouse injected with 1×50 mg/kg at 1 hr, 6 hr, and 24 hrs after injection, and the total amount.

FIG. 5: The amount of oligomers having the designs set forth in SEQ ID NO: 29 and SEQ ID NO: 30 present in the liver and kidney of mice injected with 1×50 mg/kg at 24 hrs after injection.

FIG. 6: Biodistribution/bioavailability of oligomers having the designs set forth in SEQ ID NO: 29 and SEQ ID NO: 30 present in the liver, kidney, urine and other tissues of mice injected with 50 mg/kg at 24 hrs after injection.

### 6. DETAILED DESCRIPTION

### 6.1 The Oligomer

The present invention employs oligomeric compounds (referred herein as oligomers), for use in modulating the function of nucleic acid molecules encoding mammalian Hif-1alpha, such as the Hif-1alpha encoding nucleic acid shown in SEQ ID NO: 1, and naturally occurring variants of such nucleic acid molecules encoding mammalian Hif-1alpha.

The terms “oligomer,” “oligomeric compound,” and “oligonucleotide” are used interchangeably in the context of the invention, and refer to a molecule formed by covalent linkage of two or more contiguous monomers by, for example, a phosphate group (forming a phosphodiester linkage between nucleosides) or a phosphorothioate group (forming a phosphorothioate linkage between nucleosides). The oligomer consists of, or comprises, 12-14 monomers.

In some embodiments, an oligomer comprises nucleosides, or nucleoside analogues, or mixtures thereof as referred to herein. An “LNA oligomer” or “LNA oligonucleotide” refers to an oligonucleotide containing one or more LNA monomers.

The term “monomer” includes both nucleosides and deoxynucleosides (collectively, “nucleosides”) that occur naturally in nucleic acids and that do not contain either modified sugars or modified nucleobases, i.e., compounds in which a ribose sugar or deoxyribose sugar is covalently bonded to a naturally-occurring, unmodified nucleobase (base) moiety (i.e., the purine and pyrimidine heterocycles adenine, guanine, cytosine, thymine or uracil) and “nucleoside analogues,” which are nucleosides that, either do occur naturally in nucleic acids or do not occur naturally in nucleic acids, wherein either the sugar moiety is other than a ribose or a deoxyribose sugar (such as bicyclic sugars or 2′ modified sugars, such as 2′ substituted sugars), or the base moiety is modified (e.g., 5-methylcytosine), or both.

An “RNA monomer” is a nucleoside containing a ribose sugar and an unmodified nucleobase.

A “DNA monomer” is a nucleoside containing a deoxyribose sugar and an unmodified nucleobase.

A “Locked Nucleic Acid monomer,” “locked monomer,” or “LNA monomer” is a nucleoside analogue having a bicyclic sugar, as further described herein below.

The terms “corresponding nucleoside analogue” and “corresponding nucleoside” indicate that the base moiety in the nucleoside analogue and the base moiety in the nucleoside are identical. For example, when the “nucleoside” contains a 2′-deoxyribose sugar linked to an adenine, the “corresponding nucleoside analogue” contains, for example, a modified, sugar linked to an adenine base moiety.

The terms “oligomer,” “oligomeric compound,” and “oligonucleotide” are used interchangeably in the context of the invention, and refer to a molecule formed by covalent linkage of two or more contiguous monomers by, for example, a phosphate group (forming a phosphodiester linkage between nucleosides) or a phosphorothioate group (forming a phosphorothioate linkage between nucleosides). In some embodiments, the oligomer comprises, or consists of, 12, 13, or 14 monomers. In other embodiments, such s those set forth in SEQ ID NO: 29 or SEQ ID NO: 30, the oligomer comprises, or consists of, 16 monomers.

In some embodiments, an oligomer comprises nucleosides, or nucleoside analogues, or mixtures thereof as referred to herein. An “LNA oligomer” or “LNA oligonucleotide” refers to an oligonucleotide containing one or more LNA monomers.

In various embodiments, the oligomer comprises or consists of contiguous monomers having a sequence that is identically present in SEQ ID NO: 2, SEQ ID NO: 3, SEQ ID NO: 4 or SEQ ID NO: 5.

In a particular embodiment, the oligomer consists of contiguous monomers having the sequence set forth in SEQ ID NO: 5.

It is preferred that the compound according to the invention is a linear molecule or is synthesized as a linear molecule. The oligomer is a single stranded molecule, and preferably does not comprise short regions of for example, at least 3, 4 or 5 contiguous nucleosides, which are complementary to equivalent regions within the same oligomer (i.e. duplexes)—in this regards, the oligomer is not (essentially) double stranded. In some embodiments, the oligomer is essentially not double stranded, such as is not a siRNA. In various embodiments, the oligomer of the invention consists entirely of the contiguous nucleoside region. Thus, the oligomer is not substantially self-complementary.

### 6.2 Gapmer Design

Preferably, the oligomer of the invention is a gapmer. A gapmer is an oligomer which comprises a contiguous stretch of monomers capable of recruiting an RNAse, such as RNAseH, such as a region of at least 7 DNA monomers, referred to herein in as “region B”, wherein region B is flanked both 5′ and 3′ by regions respectively referred to as regions A and C, each of regions A and C comprising or consisting of 1, 2 or 3 nucleoside analogues, such as 1, 2 or 3 affinity-enhancing nucleoside analogues.

In various embodiments, the oligomer has the design 5′-A-B-C-3′, wherein region A which consists of 1, 2 or 3 contiguous nucleoside analogues; region B consists of 7, 8, 9 or 10 nucleosides which are capable of recruiting RNaseH, such as DNA nucleosides, and, region C consists of 1, 2 or 3 contiguous nucleoside analogues. In some embodiments, the oligomer has the design 5′-A-B-C(-D) 3′, wherein region A which consists of 1, 2 or 3 contiguous nucleoside analogues, region B consists of 7, 8, 9 or 10 nucleosides which are capable of recruiting RNaseH, such as DNA nucleosides, region C consists of 1, 2 or 3 contiguous nucleoside analogues; and region D, when present, is a single DNA nucleoside.

In certain embodiments, oligomers having the design A-B-C(-D) are selected from SEQ ID NOs: 6, 7, 8, 9, 10, 11, 12, 13, 14, 15, 16 and 17 as shown in Table 1:

TABLE 1

| Motif sequence | Size | Design | Sequence (A-b-C(-d)) |
|---|---|---|---|
| SEQ ID NO: 6 | 14 | 2-9-2(-1) | 5′-GGcaagcatccTGt-3′ |
| SEQ ID NO: 7 | 14 | 2-8-3(-1) | 5′-GGcaagcatcCTGt-3′ |
| SEQ ID NO: 8 | 14 | 3-7-3(-1) | 5′-GGCaagcatcCTGt-3′ |
| SEQ ID NO: 9 | 14 | 3-8-3 | 5′-GGCaagcatccTGT-3′ |
| SEQ ID NO: 10 | 14 | 2-9-3 | 5′-GGcaagcatccTGT-3′ |
| SEQ ID NO: 11 | 13 | 2-9-2 | 5′-GGcaagcatccTG-3′ |
| SEQ ID NO: 12 | 13 | 2-8-3 | 5′-GGcaagcatgCTG-3′ |
| SEQ ID NO: 13 | 13 | 2-8-3 | 5′-GCaagcatccTGT-3′ |
| SEQ ID NO: 14 | 13 | 2-9-2 | 5′-GCaagcatcctGT-3′ |
| SEQ ID NO: 15 | 12 | 1-9-2 | 5′-GcaagcatccTG-3′ |
| SEQ ID NO: 16 | 12 | 2-8-2 | 5′-GCaagcatccTG-3′ |
| SEQ ID NO: 17 | 12 | 2-7-3 | 5′-GCaagcatcCTG-3′ |

Bold uppercase letters in Table 1 denote nucleoside analogues, such as LNA monomers, lower case letters denote nucleosides which are capable of recruiting RNAse II, such as DNA monomers. In some embodiments the linkages are all phosphorothioate. In some aspects, the cytosine bases the nucleoside analogues are each 5-methylcytosine.

In some embodiments the nucleoside analogues are LNA monomers. In various embodiments, the oligomer has the design 5′-A-B-C-3′, wherein region A which consists of 1, 2 or 3 contiguous LNA monomers, region B consists of 7, 8, 9 or 10 contiguous DNA monomers, and region C consists of 1, 2 or 3 contiguous LNA monomers. In some embodiments, the oligomer has the design 5′-A-B-C(-D)-3′, wherein region A consists of 1, 2 or 3 contiguous LNA monomers, region B consists of 7, 8, 9 or 10 contiguous DNA monomers; region C consists of 1, 2 or 3 contiguous LNA monomers, and D, if present, is a single DNA monomer.

In various embodiments, the oligomer consists of 12 contiguous monomers, wherein region A consists of 1 or 2 LNA monomers, region B consists of 8 or 9 DNA monomers, and region C consists of 1 or 2 LNA monomers.

In various embodiments, the oligomer consists of 13 or 14 contiguous monomers and has the design 5′-A-B-C-3′, wherein region A consists of 2 or 3 contiguous LNA monomers; region B consists of 8 or 9 contiguous DNA monomers, and region C consists of 2 or 3 contiguous LNA monomers.

Preferably the oligomer comprises a region having the design 5′-A-B-C-3′, wherein region A comprises least one nucleoside analogue, such as at least one LNA monomer, such as 1, 2 or 3 nucleoside analogues, such as LNA monomers, region B comprises, or consists of, at 7, 8, 9 or 10 contiguous nucleosides which are capable of recruiting RNAse (when formed in a duplex with a complementary RNA molecule, such as the mRNA target), such as DNA monomers, and region C comprises, or consists of, at least one nucleoside analogue, such as at least one LNA monomer, such as 1, 2 or 3 nucleoside analogues, such as LNA monomers.

In some embodiments, the oligomer consists of 12, 13 or 14 monomers, wherein the gapmer design is 5′-A-B-C-3′. In some embodiments, region A consists of 1 LNA monomer. In some embodiments, region A consists of 2 LNA monomers. In other embodiments, region A consists of 3 LNA monomers. In some embodiments, region C consists of 1 LNA monomer. In other embodiments, region C consists of 2 LNA monomers. In still other embodiments, region C consists of 3 LNA monomers. In some embodiments, region B consists of 7 nucleosides. In other embodiments, region B consists of 8 nucleosides. In yet other embodiments, region B consists of 9 nucleosides. In particular embodiments, region B consists of 10 nucleosides. In certain embodiments, region B consists of nucleosides that are DNA monomers.

In some embodiments, region B comprises at least one LNA monomer which is in the alpha-L configuration, such as 2, 3, 4, 5, 6, 7, 8 or 9 LNA monomers in the alpha-L-configuration. In some embodiments, region B comprises at least one alpha-L-oxy LNA unit. In certain embodiments, all LNA monomers in region B are in the alpha-L-configuration and are alpha-L-oxy LNA monomers. In certain embodiments, the number of monomers present in regions A, B and C, respectively, is selected from the group consisting of 2-8-2, 3-8-3, 2-8-3, 3-8-2, 4-8-1, 4-8-2, 1-8-4, 2-8-4, or; 1-9-1, 1-9-2, 2-9-1, 2-9-2, 2-9-3, 3-9-2, 1-9-3, 3-9-1, 4-9-1, 1-9-4, or; 1-10-1, 1-10-2, 2-10-1, 2-10-2, 1-10-3, and 3-10-1. In some embodiments, the number of monomers present in regions A, B and C, respectively, is selected from the group consisting of 3-7-3, 2-7-3, 3-7-3, 3-7-4, and 4-7-3. In some embodiment regions A and C consist of 2′-MOE RNA monomers or 2′fluoro-DNA monomers. In some embodiments each of regions A and C consist of two LNA monomers, and region B consists of 8 or 9 nucleosides, preferably DNA monomers.

In some embodiments, the oligomer is a 12mer, wherein region A consists of a single nucleoside analogue, such as LNA, region B consists of 9 nucleosides, preferably DNA nucleosides, and region C consists of 2 nucleoside analogues, preferably LNA monomers to give a gapmer with a 1-9-2 design. In some embodiments, the 12mer has a 2-8-2 design, such as a 2-8-2 design wherein regions A and C consist of LNA monomers, and region B consists of DNA monomers.

The oligomers of the invention can, for example, be selected from the group consisting of: SEQ ID NOs: 19, 20, 21, 22, 23, 24, 25, 28 and 27 as set forth in Table 2. In various embodiments, the oligomer is either SEQ ID NO: 20 or SEQ ID NO: 27.

TABLE 2

| Test | | |
|---|---|---|
| substance | Sequence | Size |
| SEQ ID | 5′-TₛGₛGₛcₛaₛaₛgₛcₛaₛtₛcₛcₛTₛGₛTₛa- | 16 |
| NO: 18 | 3′ | |
| SEQ ID | 5′-GₛGₛcₛaₛaₛgₛcₛaₛtₛcₛcₛTₛGₛt-3′ | 14 |
| NO: 19 | | |
| SEQ ID | 5′-GₛCₛaₛaₛgₛcₛaₛtₛcₛcₛTₛG-3′ | 12 |
| NO: 20 | | |
| SEQ ID | 5′-GₛGₛ^{m}CₛaₛaₛgₛcₛaₛtₛcₛcₛTₛGₛT-3′ | 14 |
| NO: 21 | | |
| SEQ ID | 5′-GₛGₛcₛaₛaₛgₛcₛaₛtₛcₛcₛTₛGₛT-3′ | 14 |
| NO: 22 | | |
| SEQ ID | 5′-GₛGₛcₛaₛaₛgₛcₛaₛtₛcₛcₛTₛG-3′ | 13 |
| NO: 23 | | |
| SEQ ID | 5′-GₛGₛcₛaₛaₛgₛcₛaₛtₛcₛ^{m}CₛTₛG-3′ | 13 |
| NO: 24 | | |
| SEQ ID | 5′-Gₛ^{m}CₛaₛaₛgₛcₛaₛtₛcₛcₛTₛGₛT-3′ | 13 |
| NO: 25 | | |
| SEQ ID | 5′-Gₛ^{m}CₛaₛaₛgₛcₛaₛtₛcₛcₛtₛGₛT-3′ | 13 |
| NO: 26 | | |
| SEQ ID | 5′-GₛcₛaₛaₛgₛcₛaₛtₛcₛcₛTₛG-3′ | 12 |
| NO: 27 | | |

Bold uppercase letters in Table 2 denote LNA monomers, preferably beta-D-oxy LNA monomers, lowercase letters denote DNA monomers, subscript “s” denotes a phosphorothioate linkage, superscript “m” before C denotes a 5-methylcytosine base.

### 6.3 Internucleoside Linkages

The monomers of the oligomers described herein are coupled together via linkage groups. Suitably, each monomer is linked to the 3′ adjacent monomer via a linkage group.

The terms “linkage group” or “internucleoside linkage” means a group capable of covalently coupling together two contiguous monomers. Specific and preferred examples include phosphate groups (forming a phosphodiester between adjacent nucleoside monomers) and phosphorothioate groups (forming a phosphorothioate linkage between adjacent nucleoside monomers).

Suitable internucleoside linkages include those listed in PCT/DK2006/000512, for example the internucleoside linkages listed on the first paragraph of page 34 of PCT/DK2006/000512 (hereby incorporated by reference).

It is, in some embodiments, preferred to modify the internucleoside linkage from its normal phosphodiester to one that is more resistant to nuclease attack, such as phosphorothioate or boranophosphate—these two, being cleavable by RNaseH, thereby permitting RNase-mediated antisense inhibition of expression of the target gene.

In certain embodiments, suitable sulphur (S) containing internucleoside linkages as provided herein are preferred. Phosphorothioate internucleoside linkages are also preferred, particularly for the gap region (B) of gapmers. In some embodiments, phosphorothioate linkages are also used in the flanking regions (A and C).

In various embodiments, regions A, B and C, comprise internucleoside linkages other than phosphorothioate, such as phosphodiester linkages, particularly, for instance when the use of nucleoside analogues protects the internucleoside linkages within regions A and C from endonuclease degradation—such as when regions A and C comprise LNA monomers.

The internucleoside linkages in the oligomer can be phosphodiester, phosphorothioate or boranophosphate so as to allow RNaseH cleavage of targeted RNA. Phosphorothioate is preferred, for improved nuclease resistance and, for example, for ease of manufacture.

In some aspects of the oligomer of the invention, the monomers are linked to each other by means of phosphorothioate groups.

It is recognized that the inclusion of phosphodiester linkages, such as one or two linkages, into an otherwise phosphorothioate oligomer, particularly between or adjacent to nucleoside analogues (typically in region A and/or C) can modify the bioavailability and/or bio-distribution of an oligomer—see, e.g., WO2008/053314, hereby incorporated by reference.

In some embodiments, such as some embodiments of the 12mer, 13mer or 14mer, the oligomer comprises a single phosphodiester bond which links monomers within regions A or C, which links the 3′-most monomer of region A to the 5′-most monomer of region B, or which links the 3″-most monomer of region B to the 5′-most monomer of region C. In certain embodiments, the remaining internucleoside linkages are all phosphorothioate linkages.

In some embodiments, such as some embodiments of the 12mer, 13mer or 14mer, the oligomer comprises two phosphodiester bonds which are positioned within or adjacent to regions A and/or C, such as between two LNA monomers within regions A and/or C. In this context, a phosphodiester linkage group is “adjacent” to region A when it links the 3′-most monomer of region A to the 5′-most monomer of region B. Likewise, phosphodiester linkage group is “adjacent” to region C when it links the 3′-most monomer of region B to the 5′-most monomer of region C, or when it links the 3″-most monomer of region C to the 5′-most monomer of region D, if present. In certain embodiments, the remaining internucleoside linkages are all phosphorothioate linkages.

In certain embodiments, such as the embodiments referred to above, where suitable and not specifically indicated, all remaining linkage groups are either phosphodiester or phosphorothioate, or a mixture thereof.

In some embodiments all the internucleoside linkage groups are phosphorothioate.

When referring to specific gapmer oligonucleotide sequences, such as those provided herein, it will be understood that, in various embodiments, when the linkages are phosphorothioate linkages, alternative linkages, such as those disclosed herein may be used, for example phosphate (phosphodiester) linkages may be used particularly for linkages between nucleoside analogues, such as LNA monomers. Likewise, in various embodiments, when referring to specific gapmer oligonucleotide sequences, such as those provided herein, when one or more monomers in region C comprises a 5-methylcytosine base, other monomers in that region may contain unmodified cytosine bases.

### 6.4 The Target

The terms “nucleic acid” and “polynucleotide” are used interchangeably herein, and are defined as a molecule formed by covalent linkage of two or more monomers, as above-described. Including 2 or more monomers, “nucleic acids” may be of any length, and the term is generic to “oligomers”, which have the lengths described herein. The terms “nucleic acid” and “polynucleotide” include single-stranded, double-stranded, partially double-stranded, and circular molecules.

In certain embodiments, oligomers described herein bind to a region of the target nucleic acid (the “target region”) by either Watson-Crick base pairing, Hoogsteen hydrogen bonding, or reversed Hoogsteen hydrogen bonding, between the monomers of the oligomer and monomers of the target nucleic acid. Such binding is also referred to as “hybridisation.” Unless otherwise indicated, binding is by Watson-Crick pairing of complementary bases (i.e., adenine with thymine (DNA) or uracil (RNA), and guanine with cytosine), and the oligomer binds to the target region because the sequence of the oligomer is identical to, or partially-identical to, the sequence of the reverse complement of the target region; for purposes herein, the oligomer is said to be “complementary” or “partially complementary” to the target region, and the percentage of “complementarity” of the oligomer sequence to that of the target region is the percentage “identity” to the reverse complement of the sequence of the target region.

Unless otherwise made clear by context, the “target region” herein will be the region of the target nucleic acid having the sequence that best aligns with the reverse complement of the sequence of the specified oligomer (or region thereof), using the alignment program and parameters described herein below.

In determining the degree of “complementarity” between oligomers of the invention (or regions thereof) and the target region of the nucleic acid which encodes mammalian Hif1-alpha, the degree of “complementarity” (also, “homology”) is expressed as the percentage identity between the sequence of the oligomer (or region thereof) and the reverse complement of the sequence of the target region that best aligns therewith. The percentage is calculated by counting the number of aligned bases that are identical as between the 2 sequences, dividing by the total number of contiguous monomers in the oligomer, and multiplying by 100. In such a comparison, if gaps exist, it is preferable that such gaps are merely mismatches rather than areas where the number of monomers within the gap differs between the oligomer of the invention and the target region.

Amino acid and polynucleotide alignments, percentage sequence identity, and degree of complementarity may be determined for purposes of the invention using the ClustalW algorithm using standard settings: see http://www.ebi.ac.uk/emboss/align/index/html, Method: EMBOSS::water (local): Gap Open=10.0, Gap extend=0.5, using Blosum 62 (protein), or DNAfull for nucleoside/nucleobase sequences.

As will be understood, depending on context, “mismatch” refers to a non-identity in sequence (as, for example, between the nucleobase sequence of an oligomer and the reverse complement of the target region to which it binds), or to noncomplementarity in sequence (as, for example, between an oligomer and the target region to which it binds).

Suitably the oligomer of the invention is capable of down-regulating, expression of a target nucleic acid, such as the Hif-1alpha gene, such as the nucleic acid having the sequence of SEQ ID NO: 1 which is the mRNA (cDNA) sequence of the human Hif-1alpha gene. In this regard, the oligomer of the invention can effect the inhibition of Hif-1alpha, typically in a mammalian such as a human cell.

In some embodiments, the oligomers of the invention bind to the target nucleic acid and effect inhibition of expression of at least 10% or 20% compared to the normal expression level, more preferably at least a 30%, 40%, 50%, 60%, 70%, 80%, 90% or 95% inhibition compared to the normal expression level. In some embodiments, such modulation is seen when using from 0.04 nM to 25 nM, such as from 0.8 nM to 20 nM concentration of the compound of the invention. In the same or a different embodiment, the inhibition of expression is less than 100%, such as less than 98% inhibition, less than 95% inhibition, less than 90% inhibition, less than 80% inhibition, such as less than 70% inhibition. In certain embodiments, modulation of expression level is determined by measuring protein levels, e.g. by the methods such as SDS-PAGE followed by western blotting using suitable antibodies raised against the target protein. Alternatively, modulation of expression levels can be determined by measuring levels of mRNA, e.g. by northern blotting or quantitative RT-PCR. When measuring via in RNA levels, the level of down-regulation when using an appropriate dosage, such as from 0.04 mM to 25 NM, such as from 0.8 nM to 20 nM concentration, is, in some embodiments, typically to a level of 10-20% the normal levels in the absence of the compound of the invention.

The invention therefore provides a method of down-regulating or inhibiting the expression of Hif-1alpha protein and/or mRNA in a cell which is expressing Hif-1alpha protein and/or mRNA, said method comprising administering the oligomer or conjugate according to the invention to said cell to down-regulating or inhibiting the expression of Hif-1 alpha protein and/or mRNA in said cell. Suitably the cell is a mammalian cell such as a human cell. In some embodiments, administration occurs in vitro. In other embodiments, administration occurs in vivo.

The term “target nucleic acid”, as used herein refers to the DNA or RNA encoding mammalian Hif-1alpha polypeptide, such as human Hif-1alpha, such as SEQ ID NO: 1. Hif-1alpha encoding nucleic acids or naturally occurring variants thereof, and RNA nucleic acids derived there from, preferably mRNA, such as pre-mRNA, although preferably mature mRNA. In some embodiments, for example when used in research or diagnostics the “target nucleic acid” is a cDNA or a synthetic oligonucleotide derived from the above DNA or RNA nucleic acid targets. The oligomer according to the invention is preferably capable of hybridising to the target nucleic acid. It will be recognized that SEQ ID NO: 1 is a cDNA sequences, and as such, corresponds to the mature mRNA target sequence, although uracil is replaced with thymidine in the cDNA sequences.

The term “naturally occurring variant thereof” refers to variants of the Hif-1alpha polypeptide of nucleic acid sequence which exist naturally within the defined taxonomic group, such as mammalian, such as mouse, monkey, and preferably human. Typically, when referring to “naturally occurring variants” of a polynucleotide the term encompasses any allelic variant of the Hif-1alpha encoding genomic DNA which are found at the Chromosome 14; Location: 14q21-q24 Mb by chromosomal translocation or duplication, and the RNA, such as mRNA derived therefrom. In certain embodiments, “naturally occurring variants” include variants derived from alternative splicing of the Hif-1alpha in RNA. When referenced to a specific polypeptide sequence, e.g., the term also includes naturally occurring forms of the protein which are processed, for example by co- or post-translational modifications such as signal peptide cleavage, proteolytic cleavage, glycosylation, etc.

### 6.5 Oligomer Sequences

In certain embodiments, the oligomer comprises, or consists of, a sequence that is fully complementary (perfectly complementary) to a target region of a nucleic acid which encodes a mammalian Hif-1alpha SEQ ID NO: 1). Preferably, the oligomer comprises or consists of a contiguous sequence which is identical to the reverse complement of a target region present in the nucleic acid having the sequence of SEQ ID NO: 1—preferably the target region of SEQ ID NO: 1 is found between (or is) residues 1198 and 1212 (inclusive). Thus, in some embodiments, the oligomer comprises, or consists of or a sequence that is identically present in SEQ ID NO: 2, 3, 4, or 5, e.g., ggcaagcatcctgt-3′ (SEQ ID NO: 2), 5′-gcaagcatcctgt-3″ (SEQ ID NO: 3), 5′-ggcaagcatcctg-3′ (SEQ ID NO: 4) or 5′-gcaagcatcctg-3′ (SEQ ID NO: 5).

Thus, in some embodiments, the oligomer comprises, or consists of, the sequence set forth in SEQ ID NOs: 2, 3, 4 or 5 (Sequence motifs) or in SEQ ID NOs: 6-16 or 17. In certain aspects, the oligomer comprises nucleosides and nucleoside analogues. In various embodiments, the oligomer comprising nucleosides and nucleoside analogues has a gapmer design such as 5′-A-B-C-3′ or 5′-A-B-C(-D)-3″ as described above.

In some embodiments, an oligomer as described herein is covalently linked to one or more moieties that are not themselves nucleic acids or monomers (“conjugated moieties”) as described further below.

In some embodiments, the oligomer according to the invention is not: 5′-GₓGₓcₛaₛaₛgₛcₛaₛtₛcₛcₛTₓGₓT-3′ or 5′-TₓTₓaₛcₛtₛgₛcₛcₛTₓTₓA-3′ or 5′-GₛGₛcₛaₛaₛgₛcₛaₛtₛcₛcₛTₛGₛt-3″ or 5′-TₛTₛaₛcₛtₛgₛcₛcₛtₛtₛcₛTₛTₛa-3′ (as disclosed in WO2006/050734) wherein uppercase letters denote an LNA monomer, such as a beta-D-oxy-LNA monomer, lowercase letters denote a 2′-deoxynucleoside monomer, an underlined letter denotes either a beta-D-oxy-LNA monomer or a 2′-deoxynucleoside, subscript “s” denotes a phosphorothioate linkage group between adjacent monomers, and subscript “x” denotes either a phosphorothioate linkage group or a phosphodiester linkage group between adjacent monomers.

In some embodiments, the sequence of the oligomer according to the invention is not 5′-GGCAAGCATCCTGT-3′ or 5″-TTACTGCCTTCTTA-3′.

### 6.6 Nucleosides and Nucleoside Analogues

The term “nucleotide” as used herein, refers to a glycoside comprising a sugar moiety, a base moiety and a covalently linked phosphate group and covers both naturally occurring nucleotides, such as DNA or RNA, preferably DNA, and non-naturally occurring nucleotides comprising modified sugar and/or base moieties, which are also referred to as “nucleotide analogues” herein.

Non-naturally occurring nucleotides include nucleotides which have modified sugar moieties, such as bicyclic nucleotides or 2′ modified nucleotides, such as 2′ substituted nucleotides.

“Nucleotide analogues” are variants of natural nucleotides, such as DNA or RNA nucleotides, by virtue of modifications in the sugar and/or base moieties. Analogues could in principle be merely “silent” or “equivalent” to the natural nucleotides in the context of the oligonucleotide, i.e. have no functional effect on the way the oligonucleotide works to inhibit target gene expression. Such “equivalent” analogues can nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell. Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213, and in Scheme 1

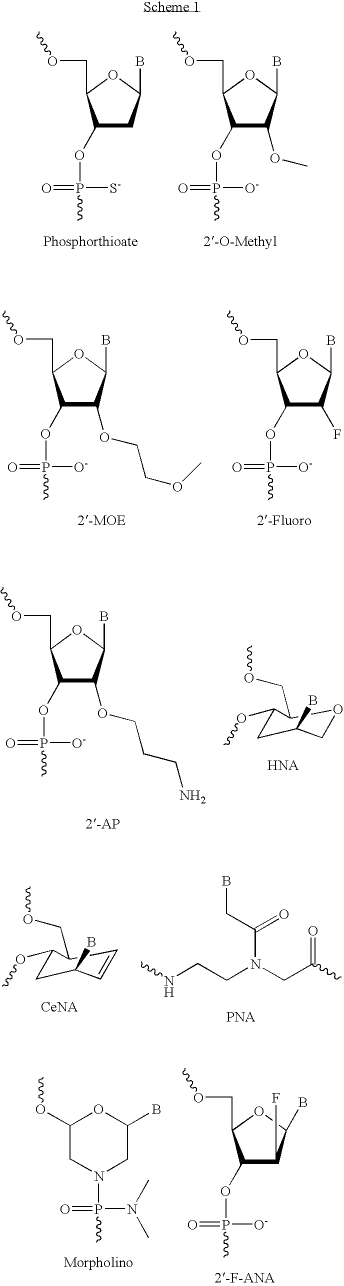

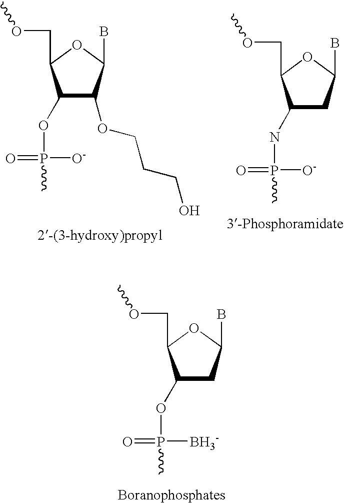

In certain embodiments, the oligomer comprises or consists of a simple sequence of naturally occurring nucleotides—preferably 2′-deoxynucleotides (referred to herein as “DNA”), but also possibly ribonucleotides (referred to herein, as “RNA”), or a combination of such, naturally occurring nucleotides and one or more non-naturally occurring nucleotides, i.e. nucleotide analogues. Such nucleotide analogues can suitably enhance the affinity of the oligomer for the target sequence.

Examples of suitable and preferred nucleotide analogues are provided by PCT/DK2006/000512 or are referenced therein.

Incorporation of affinity-enhancing nucleotide analogues in the oligomer, such as LNA or 2′-substituted sugars, can allow the size of the specifically binding oligomer to be reduced, and can also reduce the upper limit to the size of the oligomer before non-specific or aberrant binding takes place.

In some embodiments the oligomer comprises at least 2 nucleotide analogues, such as 3, 4, 5 or 6 nucleotide analogues such as LNA units. In some embodiments, the oligomer comprises a total of 3, 4 or 5 nucleotide analogues. In the by far most preferred embodiments, at least one of said nucleotide analogues is a locked nucleic acid (LNA); for example a total of 3, 4, 5 (or 6) of the nucleotide analogues can be LNA:. In some embodiments all the nucleotides analogues can be LNA.

It will be recognized that when referring to a preferred nucleotide sequence motif or nucleotide sequence, which consists of only nucleotides, the oligomers of the invention which are defined by that sequence can comprise a corresponding nucleotide analogue in place of one or more of the nucleotides present in said sequence, such as LNA monomers or other nucleotide analogues, which raise the duplex stability/Tm of the oligomer/target duplex (i.e. affinity enhancing nucleotide analogues).

Examples of such modification of the nucleotide include modifying the sugar moiety to provide a 2′-substituent group or to produce a bridged (locked nucleic acid) structure which enhances binding affinity and can also provide increased nuclease resistance.

A preferred nucleotide analogue is LNA, such as oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA). Most preferred is beta-D-oxy-LNA.

In some embodiments the nucleotide analogues present within the oligomer of the invention (such as in regions A and C mentioned herein) are independently selected from for example: 2′-O-alkyl-RNA units, 2′-amino-DNA units, T-fluoro-DNA units, LNA units, arabino nucleic acid (ANA) units, 2′-fluoro ANA units, HNA units, INA (intercalating nucleic acid—Christensen. 2001 Nucl. Acids. Res. 2002 30: 4918-4925, hereby incorporated by reference) units and 2′MOE units. In some embodiments there is only one of the above types of nucleotide analogues present in the oligomer of the invention, or contiguous nucleotide sequence thereof.

In some embodiments the nucleotide analogues are 2′-O-methoxyethyl-RNA (2′MOE), 2′-fluoro-DNA monomers or LNA nucleotide analogues, and as such the oligonucleotide of the invention comprises nucleotide analogues which are independently selected from these three types of analogue, or comprises only one type of analogue selected from the three types. In some embodiments at least one of said nucleotide analogues is 2′-MOE-RNA, such as 2, 3, 4, 5 or 6 or 2′-MOE-RNA nucleotide units. In some embodiments at least one of said nucleotide analogues is 2′-fluoro DNA, such as 2, 3, 4, 5 or 6 2′-fluoro-DNA nucleotide units. In some embodiments of the invention the oligomer is a 1-10-1, 2-8-2, 1-9-2, or 2-9-1 gapmer, where the regions A and C are either 2′MOE-RNA or 2′-fluoro-DNA.

In some embodiments, the oligomer according to the invention comprises at least one Locked Nucleic Acid (LNA) unit, such as 2, 3, 4, or 5, LNA units. In some embodiments, the oligomer comprises both beta-D-oxy-LNA, and one or more of the following LNA units: thio-LNA, amino-LNA, oxy-LNA, and/or ENA in either the beta-D or alpha-L configurations or combinations thereof. In some embodiments all LNA cytosine units are 5-methylcytosine. In some embodiments of the invention, the oligomer comprises both LNA and DNA monomers. Preferably the combined total of LNA and DNA units is 12, 13 or 14 nucleotides. In some embodiments of the invention, the nucleotide sequence of the oligomer, such as the contiguous nucleotide sequence consists of at least two or three LNA units and the remaining nucleotide units are DNA units. In some embodiments the oligomer comprises only LNA nucleotide analogues and naturally occurring nucleotides (such as RNA or DNA, most preferably DNA nucleotides), optionally with modified internucleoside linkages such as phosphorothioate.

The term “nucleobase” refers to the base moiety of a nucleotide and covers both naturally occurring a well as non-naturally occurring variants. Thus, “nucleobase” covers not only the known purine and pyrimidine heterocycles but also heterocyclic analogues and tautomeres thereof.

Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine 2-aminopurine, inosine, diaminopurine, and 2-chloro-6-aminopurine.

In some embodiments, at least one of the nucleobases present in the oligomer is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

It should be recognized that, in some aspects, the term nucleobase refers to a nucleotide which is either naturally occurring or non-naturally occurring.

### 6.6.1 LNA

The term “LNA” refers to a bicyclic nucleotide analogue, known as “Locked Nucleic Acid”. It refers to an LNA monomer, or when used in the context of an “LNA oligonucleotide” refers to an oligonucleotide containing one or more such bicyclic nucleotide analogues. Exemplary LNAs include those disclosed in International Patent Application WO 99/14226 and subsequent applications, WO0056746, WO0056748, WO00066604, WO00125248, WO0228875, WO2002094250, WO03006475 and U.S. Pat. No. 7,034,133 each of which is incorporated herein by reference in its entirety.

The LNA, used in the oligonucleotide compounds of the invention preferably has the structure of the general formula I:

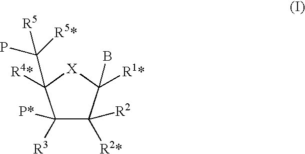

wherein X is selected from —O—, —S—, —N(RN*)-, —C(R⁶R⁶*);

B is selected from hydrogen, optionally substituted C₁₋₄-alkoxy, optionally substituted C₁₋₄-alkyl., optionally substituted C₁₋₄-acyloxy, nucleobases, DNA intercalators, photochemically active groups, thermochemically active groups, chelating groups, reporter groups, and ligands;

P designates the radical position for an internucleotide linkage to a succeeding monomer, or a 5′-terminal group, such internucleotide linkage or 5′-terminal group optionally including the substituent R⁵ or equally applicable the substituent R⁵*;

P* designates an internucleotide linkage to a preceding monomer, or a 3′-terminal group;

R⁴* and R²* together designate a biradical consisting of 1-4 groups/atoms selected from —C(R^{a}R^{b})—, —C(R)═C(R^{b}), —C(R^{a})═N, O, —Si(R^{a})₂—, S—, —SO₂—, —N(R^{a})—, and >C═Z, wherein Z is selected from —O—, —S—, and —N(R^{a})—;

and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl is optionally substituted and where two geminal substituents R^{a} and R^{b} together can be optionally substituted methylene (═CH₂), and

each of the substituents R¹*, R², R³, R⁵, R⁵, R⁵*, R⁶ and R⁶*, which are present is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂alkoxy, C₂₋₁₂-alkoxyalkyl, C₂₋₁₂-alkenyloxy, carboxy, C₁₋₁₂alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkylamino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl are optionally substituted, and where two geminal substituents together can be oxo, thioxo, imino, or optionally substituted methylene, or together can form a spiro biradical consisting of a 1-5 carbon atom(s) allylene chain which is optionally interrupted and/or terminated by one or more heteroatoms/groups selected from O, S, and —(NR^{N})—where R^{N} is selected from hydrogen and C₁₋₄-alkyl, and where two adjacent (non-geminal) substituents can designate an additional bond resulting in a double bond; and RN*, when present and not involved in a biradical, is selected from hydrogen and C1 4-alkyl; and basic salts and acid addition salts thereof;

In some embodiments R⁵* is selected from H, —CH₃, —CH₂—CH₃, —CH₂—O—CH₃, and —CH═CH₂.

In some embodiments, R⁴* and R² together designate a biradical selected from —C(R^{a}R^{b})—O, —C(R^{a}R^{b})—C(R^{c}R^{d})—O—, —C(R^{a}R^{b})—C(R^{c}R^{d})—C(R^{e}R^{f})—O, —C(R^{a}R^{b})—O—C(R^{c}R^{d})—, —C(R^{a}R^{b})—O—C(R^{c}R^{d})—O, —C(R^{a}R^{b})—C(R^{c}R^{a})—, —C(R^{a}R^{b})—C(R^{c}R^{d})—C(R^{e}R^{f})—, —C(R^{a})═C(R^{b}) —C(R^{a}R^{b})—O—c(R^{c}R^{d})—O, —C(R^{a}R^{b})—C(R^{c}R^{d})—, —C(R^{a}R^{b})—C(R^{c}R^{d})—C(R^{e}R^{f})—, —C(R^{a})═C(R^{b}) C(R^{c}R^{d})—, —C(R^{a}R^{b})—N(R^{c})—, —C(R^{a}R^{b})—C(R^{c}R^{d})—N(R^{e})—, —C(R^{a}R^{b})—N(R^{c})—O, and —C(R^{a}R^{b})— S—, —C(R^{a}R^{b})—C(R^{c}R^{d})—S—, wherein R^{a}, R^{b}, R^{c}, R^{d}, R^{e}, and R^{f} each is independently selected from hydrogen, optionally substituted C′₁₋₁₂-alkyl, optionally substituted C₂₋₁₂7 alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl are optionally substituted and where two geminal substituents R^{a} and R^{b} together can be optionally substituted methylene (═CH₂),

In a further embodiment R⁴* and R²* together designate a biradical (bivalent group) selected from —CH₂—O—, —CH₂—S—, —CH₂—NH—, —CH₂—N(CH₃)—, —CH₂—CH₂—O—, CH(CH₃)—, —CH₂—CH₂—S—, —CH₂—CH₂—CH₂—, —CH₂—CH₂—CH₂—O—, —CH₂—CH₂—CH(CH₃)—, —CH═CH—CH₂—, —CH₂—O—CH₂—O—, —CH₂—NH—O—, —CH₂—N(CH₃)—O—, —CH₂—O—CH₂—, —CH(CH₃)—O—, —CH(CH₂—O—CH₃)—O—.

In the present context, the term “optionally substituted” means that the group is substituted with 1 to 3 groups selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkoxycarbonyl, C₁₋₆-alkylcarbonyl, formyl, aryl, aryloxycarbonyl, aryloxy, arylcarbonyl, heteroaryl, heteroaryloxycarbonyl, heteroaryloxy, heteroarylcarbonyl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, sulphono, C₁₋₆-alkylsulphonyloxy, nitro, sulphanyl, C₁₋₆-alkylthio and halogen.

In the present context, the term “C₁₋₄ alkyl” means a linear or branched saturated hydrocarbon chain wherein the chain has from one to four carbon atoms, such as methyl, ethyl, n-propyl, isopropyl, n-butyl, isobutyl, sec-butyl and tert-butyl.

For all chiral centers, asymmetric groups can be found in either R or S orientation.

Preferably, the LNA used in the oligomer of the invention comprises at least one LNA unit according to any of the formulas:

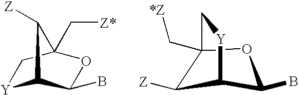

wherein Y is —O—, —O—CH₂—, —S—, —NH—, or N(R^{H}); Z and Z* are independently selected among an internucleotide linkage, a terminal group or a protecting group; B constitutes a natural or non-natural nucleotide base moiety, and R^{H} is selected from hydrogen and C₁₋₄-alkyl.

Specifically preferred LNA units are shown in Scheme 2:

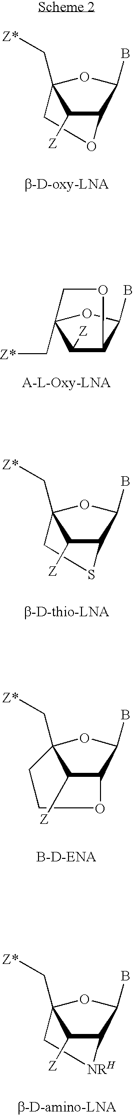

The term “thio-LNA” comprises a locked nucleotide in which Y in the general formula above is selected from S or —CH₂—S—. Thio-LNA can be in either the beta-D or alpha-L-configuration.

The term “amino-LNA” comprises a locked nucleotide in which Y in the general formula above is selected from —N(H)—, N(R)—, CH₂—N(H)—, and —CH₂—N(R)— where R is selected from hydrogen and C₁₋₄-alkyl. Amino-LNA can be in either the beta-Dor the alpha-L-configuration.

The term “oxy-LNA” comprises a locked nucleotide in which Y in the general formula above represents —O— or —CH₂—O—. Oxy-LNA can be in either the beta-D or the alpha-L-configuration.

The term “ENA” comprises a locked nucleotide in which Y in the general formula above is —CH₂—O— (where the oxygen atom of —CH₂—O— is attached to the 2′-position relative to the base B).

In a preferred embodiment the LNA monomer is selected from beta-D-oxy-LNA, alpha-L-oxy-LNA, beta-D-amino-LNA and beta-D-thio-LNA. In a particular embodiment, the LNA monomer is beta-D-oxy-INA.

### 6.7 RNAse Recruitment

It is recognized that an oligomeric compound can function via non RNase mediated degradation of target mRNA, such as by steric hindrance of translation., or other methods, however, the preferred oligomers of the invention are capable of recruiting an endoribonuclease (RNase), such as RNaseH.

It is preferable that the oligomer, or region thereof, comprises 7, 8, 9, or 10 consecutive monomers, which, when formed in a duplex with the target region of target nucleic acid (RNA) is capable of recruiting RNase. The region which is capable of recruiting RNAse can be region B as referred to in the context of a gapmer as described herein.

EP 1 222 309 provides in vitro methods for determining RNaseH activity, which may be used to determine the ability of the oligomers of the invention to recruit RNaseH. An oligomer is deemed capable of recruiting RNase H if, when contacted with the complementary region of the RNA target, it has an initial rate, as measured in pmol/l/min, of at least 1%, such as at least 5%, such as at least 10% or less than 20% of an oligonucleotide having the same base sequence but containing only DNA monomers, with no 2′ substitutions, with phosphorothioate linkage groups between all monomers in the oligonucleotide, using the methodology provided in Examples 91-95 of EP 1 222 309, incorporated herein by reference.

In some embodiments, an oligomer is deemed essentially incapable of recruiting RNaseH if, when contacted with the target region of the RNA target, and RNaseH, the RNaseH initial rate, as measured in pmol/l/min, is less than 1%, such as less than 5%, such as less than 10% or less than 20% of the initial rate determined using an oligonucleotide having the same base sequence, but containing only DNA monomers, with no 2′ substitutions, with phosphorothioate linkage groups between all monomers in the oligonucleotide, using the methodology provided in Examples 91-95 of EP 1 222 309.

In other embodiments, an oligomer is deemed capable of recruiting RNaseH if, when contacted with the target region of the RNA target, and RNaseH the RNaseH initial rate, as measured in pmol/l/min, is at least 20%, such as at least 40%, such as at least 60%, such as at least 80% of the initial rate determined using an oligonucleotide having the same base sequence, but containing only DNA monomers, with no 2′ substitutions, with phosphorothioate linkage groups between all monomers in the oligonucleotide, using the methodology provided in Examples 91-95 of EP 1 222 309.

Typically, the region of the oligomer which forms the duplex with the complementary target region of the target RNA and is capable of recruiting RNase contains DNA monomers and LNA monomers and forms a DNA/RNA-like duplex with the target region. The LNA monomers are preferably in the alpha-L configuration, particularly preferred being alpha-L-oxy LNA.

In some embodiments, the oligomer of the invention comprises both nucleosides and nucleoside analogues and can be in the form of a gapmer, a headmer or a mixmer.

A “headmer” is defined as an oligomer that comprises a first region and a second region that is contiguous thereto, with the 5′-most monomer of the second region linked to the 3′-most monomer of the first region. The first region comprises a contiguous stretch of non-RNase-recruiting nucleoside analogues, and the second region comprises a contiguous stretch (such as at least 7 contiguous monomers) of DNA monomers or nucleoside analogue monomers recognizable and cleavable by the RNAse.

A “tailmer” is defined as an oligomer that comprises a first region and a second region that is contiguous thereto, with the 5′-most monomer of the second region linked to the 3′-most monomer of the first region. The first region comprises a contiguous stretch (such as at least 7 such monomers) of DNA monomers or nucleoside analogue monomers recognizable and cleavable by the RNase, and the second region comprises a contiguous stretch of non-RNase recruiting nucleoside analogue monomers.

Other “chimeric” oligomers, called “mixmers”, consist of an alternating composition of (i) DNA monomers or nucleoside analogue monomers recognizable and cleavable by RNase, and (ii) non-RNase recruiting nucleoside analogue monomers.

In some embodiments, in addition to enhancing affinity of the oligomer for the target region, some, nucleoside analogues also mediate RNase (e.g., RNase H) binding and cleavage. Since α-L-LNA monomers recruit RNase activity to a certain extent, in some embodiments, gap regions (e.g. region B as referred to herein below) of oligomers containing α-L-LNA monomers consist of fewer monomers recognizable and cleavable by the RNase, and more flexibility in the mixmer construction is introduced.

### 6.8 Conjugates

In the context of this disclosure, the term “conjugate” indicates a compound formed by the covalent attachment (“conjugation”) of an oligomer as described herein, to one or more moieties that are not themselves nucleic acids or monomers (“conjugated moieties”). Examples of such conjugated moieties include macromolecular compounds such as proteins, fatty acid chains, sugar residues, glycoproteins, polymers, or combinations thereof. Typically proteins may be antibodies for a target protein. Typical polymers may be polyethylene glycol.

Accordingly, provided herein are conjugates comprising an oligomer as herein described, and at least one conjugated moiety that is not a nucleic acid or monomer, covalently attached to said oligomer. Therefore, in certain embodiments where the oligomer of the invention consists of contiguous monomers having a specified sequence of bases, as herein disclosed, the conjugate may also comprise at least one conjugated moiety that is covalently attached to the oligomer.

In various embodiments of the invention, the oligomer is conjugated to a moiety that increases the cellular uptake of oligomeric compounds. WO2007/031091 provides suitable ligands and conjugates, which are hereby incorporated by reference.

In various embodiments, conjugation (to a conjugated moiety) may enhance the activity, cellular distribution or cellular uptake of the oligomer of the invention. Such moieties include, but are not limited to, antibodies, polypeptides, lipid moieties such as a cholesterol moiety, cholic acid, a thioether, e.g. Hexyl-s-tritylthiol, a thiocholesterol, an aliphatic chain, e.g. dodecandiol or undecyl residues, a phospholipids, e.g., di-hexadecyl-rac-glycerol or triethylammonium 1,2-di-o-hexadecyl-rac-glycero-3-h-phosphonate, a polyamine or a polyethylene glycol chain, an adamantane acetic acid, a palmityl moiety, an octadecylamine or hexylamino-carbonyl-oxycholesterol moiety.

In certain embodiments, the oligomers of the invention are conjugated to active drug substances, for example, aspirin, ibuprofen, a sulfa drug, an antidiabetic, an antibacterial or an antibiotic.

In certain embodiments the conjugated moiety is a sterol, such as cholesterol.

In various embodiments, the conjugated moiety comprises or consists of a positively charged polymer, such as a positively charged peptides of for example 1-50, such as 2-20 such as 3-10 amino acid residues in length, and/or polyalkylene oxide such as polyethylene glycol (PEG) or polypropylene glycol—see WO 2008/034123, hereby incorporated by reference. Suitably the positively charged polymer, such as a polyalkylene oxide may be attached to the oligomer of the invention via a linker such as the releasable linker described in WO 2008/034123.

By way of example, the following moieties may be used in the conjugates of the invention:

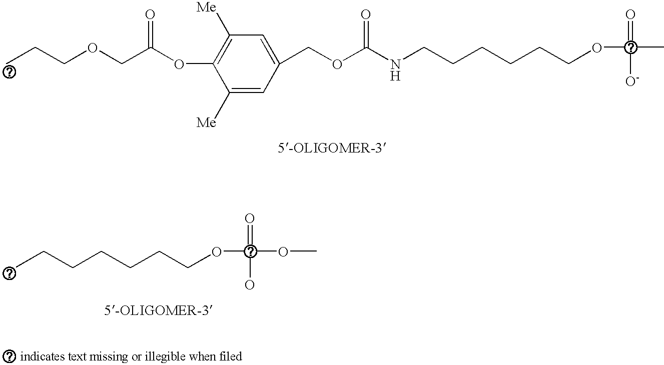

### 6.9 Activated Oligomers

The term “activated oligomer,” as used herein, refers to an oligomer of the invention that is covalently linked (i.e., functionalized) to at least one functional moiety that permits covalent linkage of the oligomer to one or more conjugated moieties, i.e., moieties that are not themselves nucleic acids or monomers, to form the conjugates herein described. Typically, a functional moiety will comprise a chemical group that is capable of covalently bonding to the oligomer via, e.g., a 3′-hydroxyl group or the exocyclic NH₂ group of the adenine base, a spacer that is preferably hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such, as those described in “Protective Groups in Organic Synthesis” by Theodora W Greene and Peter G M Wuts, 3rd edition (John Wiley & Sons, 1999). Examples of suitable hydroxyl protecting groups include esters such as acetate ester, aralkyl groups such as benzyl, diphenylmethyl, or triphenylmethyl, and tetrahydropyranyl. Examples of suitable amino protecting groups include benzyl, alpha-methylbenzyl, diphenylmethyl, triphenylmethyl, benzyloxycarbonyl, tert-butoxycarbonyl, and acyl groups such as trichloroacetyl or trifluoroacetyl. In some embodiments, the functional moiety is self-cleaving. In other embodiments, the functional moiety is biodegradable. See e.g., U.S. Pat. No. 7,087,229, which is incorporated by reference herein in its entirety.

In some embodiments, oligomers of the invention are functionalized at the 5′ end in order to allow covalent attachment of the conjugated moiety to the 5′ end of the oligomer. In other embodiments, oligomers of the invention can be functionalized at the 3′ end. In still other embodiments, oligomers of the invention can be functionalized along the backbone or on the heterocyclic base moiety. In yet other embodiments, oligomers of the invention can be functionalized at more than one position independently selected from the 5′ end, the 3′ end, the backbone and the base.

In some embodiments, activated oligomers of the invention are synthesized by incorporating during the synthesis one or more monomers that is covalently attached to a functional moiety. In other embodiments, activated oligomers of the invention are synthesized with monomers that have not been functionalized, and the oligomer is functionalized upon completion of synthesis. In some embodiments, the oligomers are functionalized with a hindered ester containing an aminoalkyl linker, wherein the alkyl portion has the formula (CH₂)_{w}, wherein w is an integer ranging from 1 to 10, preferably about 6, wherein the alkyl portion of the alkylamino group can be straight chain or branched chain, and wherein the functional group is attached to the oligomer via an ester group (—O—C(O)—(CH₂)_{w}NH).

In other embodiments, the oligomers are functionalized with a hindered ester containing a (CH₂)_{w}-sulfhydryl (SH) linker, wherein w is an integer ranging from 1 to 10, preferably about 6, wherein the alkyl portion of the alkylamino group can be straight chain or branched chain, and wherein the functional group attached to the oligomer via an ester group (—O—C(O)—(CH₂)_{w}SH)

In some embodiments, sulfhydryl-activated oligonucleotides are conjugated with polymer moieties such as polyethylene glycol or peptides (via formation of a disulfide bond).

Activated oligomers containing hindered esters as described above can be synthesized by any method known in the art, and in particular by methods disclosed in PCT Publication No. WO 2008/034122 and the examples therein, which is incorporated herein by reference in its entirety.

In still other embodiments, the oligomers of the invention are functionalized by introducing sulfhydryl, amino or hydroxyl groups into the oligomer by means of a functionalizing reagent substantially as described in U.S. Pat. Nos. 4,962,029 and 4,914,210, i.e., a substantially linear reagent having a phosphoramidite at one end linked through a hydrophilic spacer chain to the opposing end which comprises a protected or unprotected sulfhydryl, amino or hydroxyl group. Such reagents primarily react with hydroxyl groups of the oligomer. In some embodiments, such activated oligomers have a functionalizing reagent coupled to a 5′-hydroxyl group of the oligomer. In other embodiments, the activated oligomers have a functionalizing reagent coupled to a 3′-hydroxyl group. In still other embodiments, the activated oligomers of the invention have a functionalizing reagent coupled to a hydroxyl group on the backbone of the oligomer. In yet further embodiments, the oligomer of the invention is functionalized with more than one of the functionalizing reagents as described in U.S. Pat. Nos. 4,962,029 and 4,914,210, incorporated herein by reference in their entirety. Methods of synthesizing such functionalizing reagents and incorporating them into monomers or oligomers are disclosed in U.S. Pat. Nos. 4,962,029 and 4,914,210.

In some embodiments, the 5′-terminus of a solid-phase bound oligomer is functionalized with a dienyl phosphoramidite derivative, followed by conjugation of the deprotected oligomer with, e.g., an amino acid or peptide via a Diels-Alder cycloaddition reaction.

In various embodiments, the incorporation of monomers containing 2′-sugar modifications, such as a 2′-carbamate substituted sugar or a 2′-(O-pentyl-N-phthalimido)-deoxyribose sugar into the oligomer facilitates covalent attachment of conjugated moieties to the sugars of the oligomer. In other embodiments, an oligomer with an amino-containing linker at the 2′-position of one or more monomers is prepared using a reagent such as, for example, 5′-dimethoxytrityl-2′-O-(e-phthalimidylaminopentyl)-2′-deoxyadenosine-3′-N,N-diisopropyl-cyanoethoxy phosphoramidite. See, e.g., Manoharan, et al. Tetrahedron Letters, 1991, 34, 7171.

In still further embodiments, the oligomers of the invention have amine-containing functional moieties on the nucleobase, including OD the N6 purine amino groups, on the exocyclic N2 of guanine, or on the N4 or 5 positions of cytosine. In various embodiments, such functionalization is achieved using a commercial reagent that is already functionalized in the oligomer synthesis.

Some functional moieties are commercially available, for example, heterobifunctional and homobifunctional linking moieties are available from the Pierce Co. (Rockford, Ill.). Other commercially available linking groups are 5′-Amino-Modifier C6 and 3′-Amino-Modifier reagents, both available from Glen Research Corporation (Staling, Va.). 5′-Amino-Modifier C6 is also available from ABI (Applied Biosystems Inc., Foster City, Calif.) as Aminolink-2, and 3′-Amino-Modifier is also available from Clontech Laboratories Inc. (Palo Alto, Calif.). In some embodiments In some embodiments in some embodiments In some embodiments

### 6.10 Compositions

The oligomer of the invention can be used in pharmaceutical formulations and compositions. Suitably, such compositions comprise a pharmaceutically acceptable diluent, carrier, salt or adjuvant. PCT/DK2006/000512 provides suitable and preferred pharmaceutically acceptable diluent, carrier and adjuvants—which are hereby incorporated by reference. Suitable dosages, formulations, administration routes, compositions, dosage forms, combinations with other therapeutic agents, pro-drug formulations are also provided in PCT/DK2006/000512—which are also hereby incorporated by reference. Details on techniques for formulation and administration also may be found in the latest edition of “REMINGTON′S PHARMACEUTICAL SCIENCES” (Maack Publishing Co, Easton Pa.).

In some embodiments, an oligomer of the invention is covalently linked to a conjugated moiety to aid in delivery of the oligomer across cell membranes. An example of a conjugated moiety that aids in delivery of the oligomer across cell membranes is a lipophilic moiety, such as cholesterol. In various embodiments, an oligomer of the invention is formulated with lipid formulations that form liposomes, such as Lipofectamine 2000 or Lipofectamine RNAiMAX, both of which are commercially available from Invitrogen. In some embodiments, the oligomers of the invention are formulated with a mixture of, one or more lipid-like non-naturally occurring small molecules (“lipidoids”). Libraries of lipidoids can be synthesized by conventional synthetic chemistry methods and various amounts and combinations of lipidoids can be assayed in order to develop a vehicle for effective delivery of an oligomer of a particular size to the targeted tissue by the chosen route of administration. Suitable lipidoid libraries and compositions can be found, for example in Akinc et al. (2008) Nature Biotechnol., available at http://www.nature.com/ribtijournallvaop/ncutTent/abs/nbt1402.html, which is incorporated by reference herein.

As used herein, the term “pharmaceutically acceptable salts” refers to salts that retain the desired biological activity of the herein identified compounds and exhibit acceptable levels of undesired toxic effects. Non-limiting examples of such salts can be formed with organic amino acid and base addition salts formed with metal cations such as zinc, calcium, bismuth, barium, magnesium, aluminum, copper, cobalt, nickel, cadmium, sodium, potassium, and the like, or with a cation formed from ammonia, N,N′-dibenzylethylene-diamine, D-glucosamine, tetraethylammonium, or ethylenediamine; or (c) combinations of (a) and (b), e.g., a zinc tannate salt or the like.

In certain embodiments, the pharmaceutical compositions according to the invention comprise other active ingredients in addition to an oligomer or conjugate of the invention, including active agents useful for the treatment of cancer.

### 6.11 Diagnostic Applications and Methods of Treatment

The oligomers of the invention can be utilized as research reagents for, e.g., diagnostics, therapeutics and prophylaxis.

In research, such oligomers can be used to specifically inhibit the synthesis of Hif-1alpha protein (typically by degrading or inhibiting the mRNA and thereby preventing translation) in cells and experimental animals, thereby facilitating functional analysis of the target or an appraisal of its usefulness as a target for therapeutic intervention.

For diagnostic applications, the oligomers described herein can be used to detect and quantitate Hif-1alpha expression in cells and tissues by northern blotting, in-situ hybridisation or similar techniques.

For therapeutics applications, an animal or a human suspected of having a disease or disorder which can be treated by modulating the expression of Hif-1alpha can be treated by administering an effective amount of an oligomeric compound, conjugate or pharmaceutical composition in accordance with this invention. Further provided are methods of treating a mammal, such as a human, suspected of having or being prone to a disease or condition associated with abnormal expression of Hif-1alpha by administering a therapeutically or prophylactically effective amount of an oligomer, conjugate or pharmaceutical composition of the invention.

The terms “treat,” “treating” or “treatment” as used herein refer to both treatment of an existing disease (e.g., a disease or disorder as referred to herein below), or prevention of a disease, i.e., prophylaxis. It will therefore be recognized that, in certain embodiments, “treatment” includes prophylaxis.

The invention also provides for the use of the compound or conjugate of the invention as described herein for the manufacture of a medicament for the treatment of a disorder as referred to herein, or for a method of the treatment of as a disorder as referred to herein.

The compositions and conjugates described herein can be used for the treatment of conditions associated with abnormal levels of Hif-1alpha, such as hyperproliferative disorders, such as cancer. In certain embodiments, the compositions and conjugates described herein can be used, for the treatment of disorders associated with Hif-1alpha, such as artherosclerosis, psoriasis, diabetic retinopathy, macular degeneration, rheumatoid arthritis, asthma, inflammatory bowel disease, warts, allergic dermatitis, inflammation, and skin inflammation. It will be recognized that the Hif-1alpha targeting oligomers can be combined with one or more additional therapeutic agents in a pharmaceutical composition according to the invention—such as therapeutic agents provided in WO2006/050734—hereby incorporated by reference.

In some embodiments the compositions and conjugates are used for the treatment of cancer selected from kidney cancer and liver cancer.

The invention further provides for use of a compound of the invention in the manufacture of a medicament for the treatment of a disease, disorder or condition as described herein.

Generally stated, in some aspects, the invention is directed to a method of treating a mammal suffering from or susceptible to a condition associated with abnormal levels of Hif-1alpha, comprising administering to the mammal a therapeutically effective amount of an oligomer targeted to Hif-1alpha that comprises one or more LNA monomers.

An interesting aspect of the invention is directed to the use of an oligomer as defined herein or a conjugate as defined herein for the preparation of a medicament for the treatment of a disease, disorder or condition as described herein.

In some embodiments, the invention is directed to a method for treating abnormal levels of Hif-1alpha in a subject, said method comprising administering to the subject an oligomer of the invention, or a conjugate thereof or a pharmaceutical composition of the invention.

The invention also relates to an oligomer, a composition or a conjugate as defined herein for use as a medicament.

### 7. EXAMPLES

### 7.1 Example 1

### Monomer Synthesis

LNA nucleoside analogue building blocks (e.g. β-D-oxy-LNA, β-D-thio-LNA, β-D-amino-LNA and α-L-oxy-LNA) can be prepared following established published Procedures—for example see WO2007/031081 hereby incorporated by reference.

### 7.2 Example 2

### Oligonucleotide Synthesis

Oligonucleotides were synthesized according to the method described and referenced in WO 07/031,081. Beta-D-oxy LNA monomers were used.

### 7.3 Example 3

### Measurements of mRNA Levels

Antisense modulation of Hif-1alpha expression can be assayed in a variety of ways known in the art. For example, Hif-1alpha in RNA levels can be quantitated by, e.g., Northern blot analysis, competitive polymerase chain reaction (PCR), or real-time PCR. Real-time quantitative PCR is presently preferred. RNA analysis can be performed on total cellular RNA or mRNA. Methods of RNA isolation and RNA analysis such as Northern blot analysis are routine in the art and are taught in, for example, Current Protocols in Molecular Biology, John Wiley and Sons.

Real-time quantitative PCR can be conveniently accomplished using the iQ Multi-Color Real Time PCR Detection System available from BioRAD. Real-time quantitative PCR is a technique well known in the art and is taught in for example Heid et al. Real time quantitative PCR, Genome Research (1996), 6: 986-994.

### 7.4 Example 4

### Different Length (16-Mer-12 Mer) of Oligonucleotides Targeting Hif-1 Alpha mRNA (Dosing 3*5 mg/kg i.v. Three Consecutive Days) in Kidney

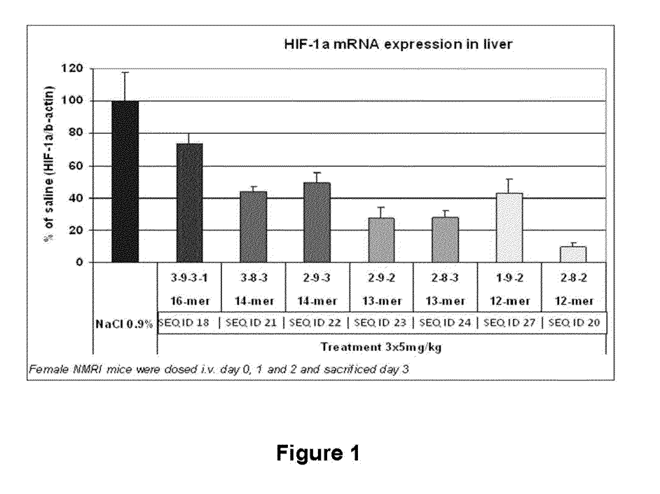

In this study 5 ing/kg/dose were administered on 3 consecutive clays (one dose/day i.v.) and animals were sacrificed 24 hours after last dosing. At sacrifice, liver was sampled. RNA was isolated from the liver and the expression of Hif-1alpha was measured using qPCR. The results are shown in FIG. 1.

### 7.5 Example 5

### Different Length (16-Mer-12 Mer) of Oligonucleotides Targeting Hif 1-Alpha mRNA (Dosing 3*5 mg/kg i.v. Three Consecutive Days) in Kidney

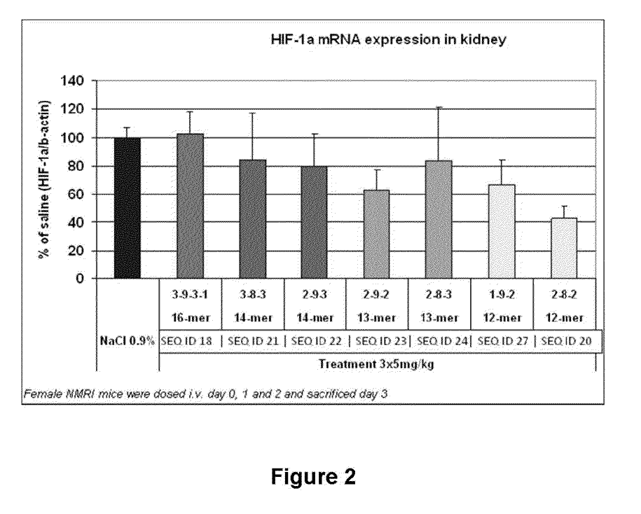

In this study 5 mg/kg/dose were dosed to NMRI mice on 3 consecutive days (one dose/day i.v.) and animals were sacrificed 24 hours after last dosing. At sacrifice, liver and kidney tissue were sampled. RNA was isolated from the tissues and the expression of Hif-1alpha mRNA was measured using qPCR. The results are shown in FIG. 2. The results were less dramatic than those seen in the liver, which is likely due to the difficulty in achieving potent knockdown of HIF-1alpha mRNA throughout the kidney (both in medulla and cortex) because oligonucleotides typically only penetrate into the cortex. The use of shortmers such as the 12mers described herein provided an improved efficacy of Hif-1alpha down-regulation in the kidney, which may be due to an ability of the oligomers to penetrate the medulla, and/or to the enhanced efficacy of the specific shortmer designs, particularly the 2-8-2 design.

### 7.6 Example 6

### Comparison of Biodistribution of Fully Phosphorothioate Gapmer with Equivalent Oligomer where Two Phosphorothioate Linkages have been Replaced with Phosphodiester

### 7.6.1 Oligonucleotide Compounds

The oligomers set forth in Table 3 were synthesized:

TABLE 3

| Sequence | | |
|---|---|---|
| Identifier | Target | Sequence |
| SEQ ID | Hif-1alpha | TGGCAAGCATCCTGTA |
| NO: 28 | | (Motif sequence) |
| SEQ ID | Hif-1alpha | 5′-TSG_{S}G_{S}c_{S}a_{S}a_{S}g_{S}c_{S}a_{S}t_{S}c_{S}c_{S} |
| NO: 29 | | T_{S}G_{S}T_{S}a-3′ |
| SEQ ID | Hif-1alpha | 5′-T_{S}GG_{S}c_{S}a_{S}a_{S}g_{S}c_{S}a_{S}t_{S}c_{S}c_{S} |
| NO: 30 | | TG_{S}T_{S}a-3′ |

In Table 3, bold uppercase letters denote beta-D-oxy LNA monomers, lowercase letters denote DNA monomers, subscript “s” denotes a phosphorothioate linkage, and the absence of a subscript “s” denotes a phosphodiester linkage.

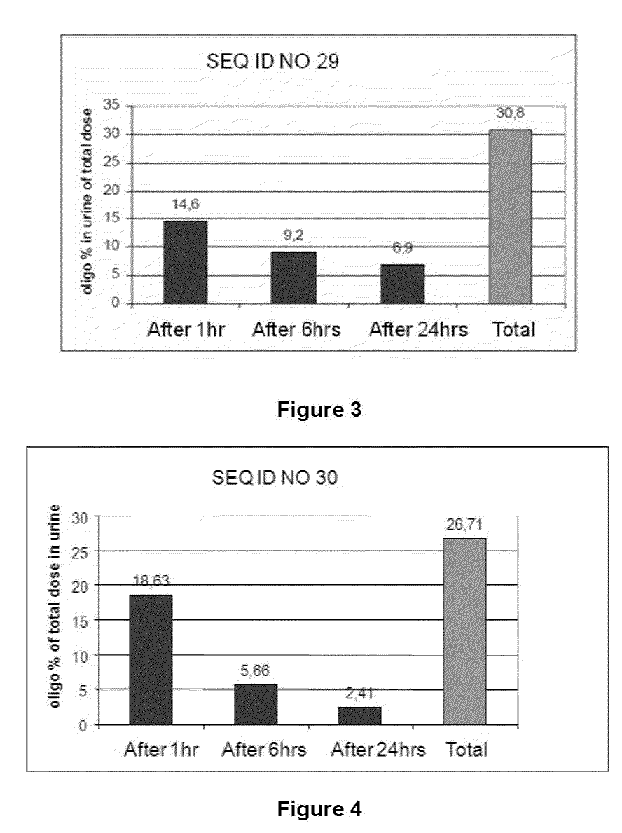

The oligomers having the designs set forth in SEQ ID NOs: 29 and 30 were injected into mice at a dosage of 50 mg/kg. Urine was sampled after 1 hour, 6 hours and 24 hours. Animals were killed after 24 hours, and the amount of each oligomer present in the liver and kidney was assessed. The results are shown in FIGS. 3, 4, 5 and 6.

The oligomer having the design shown in SEQ ID NO: 29 was found to be excreted at a slightly higher rate in the urine over the 24-hour period, although the initial rate of excretion appeared to be higher for the oligomer having the design shown in SEQ ID NO: 30. (FIGS. 3, 4).

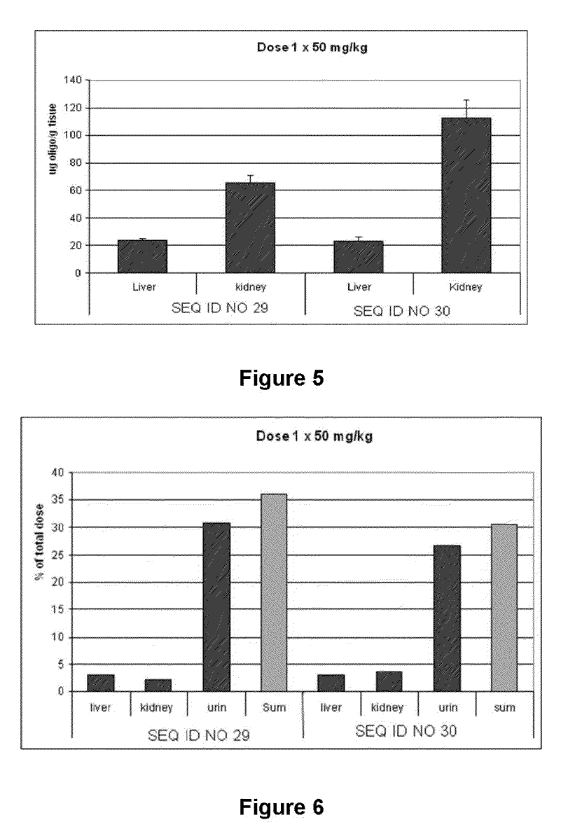

The amount of the oligomer having the design shown in SEQ ID NO: 30 with 2 PO's distributed to the kidney was found to be almost twice as much as that of the oligomer having the design shown in SEQ ID NO: 29. (FIG. 5).

The oligomer having the design of SEQ ID NO: 30 showed a wider biodistribution to other tissues—69% of the oligomer with the design set forth in SEQ ID NO: 30 distributed to other tissues as compared to 64% of the oligomer with the design set forth in SEQ ID NO: 29.

As the presence of a single phosphodiester linkage resulted in an enhanced efficacy of down-regulation of Hif-1alpha mRNA in kidney and improved biodistribution, the following oligonucleotides shown in Table 4 were designed with the aim to further enhance the activity of the shortmers in kidney:

TABLE 4

| Sequence | | |
|---|---|---|
| Identifier | Sequence | Size |
| SEQ ID NO: 31 | 5′-GCₛaₛaₛgₛcₛaₛtₛcₛcₛTₛG-3′ | 12 |
| SEQ ID NO: 32 | 5′-GCₛaₛaₛgₛcₛaₛtₛcₛcₛTG-3′ | 12 |
| SEQ ID NO: 33 | 5′-GₛcₛaₛaₛgₛcₛaₛtₛcₛcₛTG-3′ | 12 |

In Table 4, bold uppercase letters denote beta-D-oxy LNA monomers, lowercase letters denote DNA monomers, subscript “s” denotes a phosphorothioate linkage, and the absence of a subscript “s” denotes a phosphodiester linkage.

### 8. SPECIFIC EMBODIMENTS, CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. An oligonucleotide compound consisting of 12 to 16 contiguous monomers,
wherein adjacent monomers are covalently linked by a phosphate group or a phosphorothioate group,
wherein said oligomer comprises a first region of 12 contiguous monomers;
wherein at least one monomer of said first region is a nucleoside analogue; and
wherein the sequence of said first region is 5′GCAAGCATCCTG-3′ (SEQ ID NO: 5).

2. The oligomer according to claim 1, wherein each nucleoside analogue is independently selected from the group consisting of an LNA monomer, a monomer containing a 2′-O-alkyl-ribose sugar, a monomer containing a 2′-O-methyl-ribose sugar, a monomer containing a 2′-amino-deoxyribose sugar, and a monomer containing a 2′ fluoro-deoxyribose sugar.

3. The oligomer according to claim 2, wherein the nucleoside analogue is an LNA monomer.

4. The oligomer according to claim 1, wherein the oligomer is a gapmer, and wherein said gapmer comprises from the 5′ end to the 3′ end:
(i) a region A consisting of from 1 to 3 contiguous monomers, wherein at least one monomer is a nucleoside analogue,
(ii) a region B, the 5′ end of which is covalently linked to the 3′ end of region A and consisting of from 8 to 9 contiguous monomers, wherein at least one monomer is a nucleoside; and
(iii) a region C, the 5′ end of which is covalently linked to the 3′ end of region B and consisting of from 1 to 3 contiguous monomers, wherein at least one monomer is a nucleoside analogue.

5. The oligomer according to claim 1, wherein the oligomer is a gapmer, and wherein said gapmer comprises from the 5′ end to the 3′ end:
(i) a region A consisting of from 1 to 3 contiguous monomers, wherein at least one monomer is a nucleoside analogue,
(ii) a region B, the 5′ end of which is covalently linked to the 3′ end of region A and consisting of from 8 to 9 contiguous monomers, wherein at least one monomer is a nucleoside;
(iii) a region C, the 5′ end of which is covalently linked to the 3′ end of region B and consisting of from 1 to 3 contiguous monomers, wherein at least one monomer is a nucleoside analogue; and
(iv) a region D, the 5′ end of which is convalently linked to the 3′ end of region C and consisting of 1 monomer, which is a nucleoside.

6. The oligomer according to claim 4, wherein the compound is selected from

| | |
|---|---|
| 5′-Gₛ^{m}CₛaₛaₛgₛcₛaₛtₛcₛcₛTₛG-3′; | (SEQ ID NO 20) |
| and | |
| 5′-GₛcₛaₛaₛgₛcₛaₛtₛcₛcₛTₛG-3′; | (SEQ ID NO 27) |

wherein bold uppercase letters denote LNA monomers, lowercase letters denote DNA monomers, subscript “s” denotes a phosphorothioate linkage, and “^{m}C” denotes a 5-methylcytosine base.

7. The oligomer according to claim 4, wherein the compound is selected from

| | |
|---|---|
| 5′-GₛGₛ^{m}CₛaₛaₛgₛcₛaₛtₛcₛcₛTₛGₛT-3′; | (SEQ ID NO: 21) |
| 5′-GₛGₛcₛaₛaₛgₛcₛaₛtₛcₛcₛTₛGₛT-3′; | (SEQ ID NO: 22) |
| 5′-GₛGₛcₛaₛaₛgₛcₛaₛtₛcₛcₛTₛG-3′; | (SEQ ID NO: 23) |
| 5′-GₛGₛcₛaₛaₛgₛcₛaₛtₛcₛ^{m}CₛTₛG-3′; | (SEQ ID NO: 24) |
| 5′-Gₛ^{m}CₛaₛaₛgₛcₛaₛtₛcₛcₛTₛGₛT-3′; | (SEQ ID NO: 25) |
| 5′-Gₛ^{m}CₛaₛaₛgₛcₛaₛtₛcₛcₛtₛGₛT-3′; | (SEQ ID NO: 26) |
| 5′-GCₛaₛaₛgₛcₛaₛtₛcₛcₛTₛG-3′; | (SEQ ID NO: 31) |
| 5′-GCₛaₛaₛgₛcₛaₛtₛcₛcₛTG-3′; | (SEQ ID NO: 32) |
| and | |
| 5′-GₛcₛaₛaₛgₛcₛaₛtₛcₛcₛTG-3′; | (SEQ ID NO: 33), |

wherein bold uppercase letters denote LNA monomers, lowercase letters denote DNA monomers, subscript “s” denotes a phosphorothioate linkage, the absence of “s” between two monomers designates a phosphodiester linkage, and “^{m}C” denotes a 5-methylcytosine base.

8. The oligomer according to claim 4, wherein the compound is selected from:

| | |
|---|---|
| 5′-GGCaagcatccTGT-3′; | (SEQ ID NO: 9) |
| 5′-GGcaagcatccTGT-3′; | (SEQ ID NO: 10) |
| 5′-GGcaagcatccTG-3′; | (SEQ ID NO: 11) |
| 5′-GGcaagcatgCTG-3′; | (SEQ ID NO: 12) |
| 5′-GCaagcatccTGT-3′; | (SEQ ID NO: 13) |
| 5′-GCaagcatccTGT-3′; | (SEQ ID NO: 14) |
| 5′-GcaagcatccTG-3′; | (SEQ ID NO: 15) |
| 5′-GCaagcatccTG-3′; | (SEQ ID NO: 16) |
| and | |
| 5′-GCaagcatccTG-3′; | (SEQ ID NO: 17) |

wherein bold uppercase letters denote nucleoside analogue monomers and lowercase letters denote nucleoside monomers.

9. The oligomer according to claim 5, wherein the compound is selected from:

| | |
|---|---|
| 5′-GGcaagcatccTGt-3′; | (SEQ ID NO: 6) |
| 5′-GGcaagcatcCTGt-3′; | (SEQ ID NO: 7) |
| and | |
| 5′-GGCaagcatcCTGt-3′; | (SEQ ID NO: 8) |

wherein bold uppercase letters denote nucleoside analogue monomers and lowercase letters denote nucleoside monomers.

10. The oligomer according to claim 8, wherein all nucleoside analogue monomers are LNA monomers, all linkages between adjacent monomers are phosphorothioate linkages and all cytosine bases in the nucleoside analogues are 5-methylcytosine.

11. The oligomer according to claim 9, wherein all nucleoside analogue monomers are LNA monomers, all linkages between adjacent monomers are phosphorothioate linkages and all cytosine bases in the nucleoside analogues are 5-methylcytosine.

12. The oligomer according to claim 5, wherein the compound is selected from
(SEQ ID NO: 18)
5′-TₛGₛGₛcₛaₛaₛgₛcₛaₛtₛcₛcₛTₛGₛTₛa-3′;
(SEQ ID NO: 19)
5′-GₛGₛcₛaₛaₛgₛcₛaₛtₛcₛcₛTₛGₛt-3′;
and
(SEQ ID NO: 30)
5′-TₛGGₛcₛaₛaₛgₛcₛaₛtₛcₛcₛTGₛTₛa-3′;
wherein bold uppercase letters denote LNA monomers, lowercase letters denote DNA monomers, subscript “s” denotes a phosphorothioate linkage, and the absence of “s” between two monomers designates a phosphodiester linkage.

13. A conjugate comprising the oligomer according to claim 1, and at least one non-nucleotide or non-polynucleotide moiety covalently attached to said oligomer.

14. A pharmaceutical composition comprising the oligomer according to claim 1 and a pharmaceutically acceptable diluent, carrier, salt or adjuvant.

15. A pharmaceutical composition comprising the conjugate according to claim 13 and a pharmaceutically acceptable diluent, carrier, salt or adjuvant.

16. A method of inhibiting the expression of Hif-1alpha in a cell, comprising contacting said cell with an effective amount of an oligomer consisting of 12 to 16 contiguous monomers,
wherein adjacent monomers are covalently linked by a phosphate group or a phosphorothioate group,
wherein said oligomer comprises a first region of 12 contiguous monomers;
wherein at least one monomer of said first region is a nucleoside analogue; and
wherein the sequence of said first region is 5′-GCAAGCATCCTG-3′ (SEQ II) NO: 5).

17. A method of inhibiting the expression of Hif-1alpha in a cell, comprising contacting said cell with an effective amount of a conjugate according to claim 13.

18. A method of inhibiting the expression of Hif-1alpha in a tissue of a mammal, comprising contacting said tissue with an effective amount of an oligomer consisting of 12 to 16 contiguous monomers,
wherein adjacent monomers are covalently linked by a phosphate group or a phosphorothioate group,
wherein said oligomer comprises a first region of 12 contiguous monomers;
wherein at least one monomer of said first region is a nucleoside analogue; and
wherein the sequence of said first region is 5′-GCAAGCATCCTG-3′ (SEQ ID NO: 5).


SEQUENCE LISTING (33 sequences)

SEQ ID NO: 1 - DNA, 3958 bp, Homo sapiens
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggaaaaaaaaaaaaaaaaaaa

SEQ ID NO: 2 - DNA, 14 bp, Artificial sequence
  Sequence motif, design or oligomer
ggcaagcatcctgt

SEQ ID NO: 3 - DNA, 13 bp, Artificial sequence
  Sequence motif, design or oligomer
gcaagcatcctgt

SEQ ID NO: 4 - DNA, 13 bp, Artificial sequence
  Sequence motif, design or oligomer
ggcaagcatcctg

SEQ ID NO: 5 - DNA, 12 bp, Artificial sequence
  Sequence motif, design or oligomer
gcaagcatcctg

SEQ ID NO: 6 - DNA, 14 bp, Artificial sequence
  Sequence motif, design or oligomer
ggcaagcatcctgt

SEQ ID NO: 7 - DNA, 14 bp, Artificial sequence
  Sequence motif, design or oligomer
ggcaagcatcctgt

SEQ ID NO: 8 - DNA, 14 bp, Artificial sequence
  Sequence motif, design or oligomer
ggcaagcatcctgt

SEQ ID NO: 9 - DNA, 14 bp, Artificial sequence
  Sequence motif, design or oligomer
ggcaagcatcctgt

SEQ ID NO: 10 - DNA, 14 bp, Artificial sequence
  Sequence motif, design or oligomer
ggcaagcatcctgt

SEQ ID NO: 11 - DNA, 13 bp, Artificial sequence
  Sequence motif, design or oligomer
ggcaagcatcctg

SEQ ID NO: 12 - DNA, 13 bp, Artificial sequence
  Sequence motif, design or oligomer
ggcaagcatgctg

SEQ ID NO: 13 - DNA, 13 bp, Artificial sequence
  Sequence motif, design or oligomer
gcaagcatcctgt

SEQ ID NO: 14 - DNA, 13 bp, Artificial sequence
  Sequence motif, design or oligomer
gcaagcatcctgt

SEQ ID NO: 15 - DNA, 12 bp, Artificial sequence
  Sequence motif, design or oligomer
gcaagcatcctg

SEQ ID NO: 16 - DNA, 12 bp, Artificial sequence
  Sequence motif, design or oligomer
gcaagcatcctg

SEQ ID NO: 17 - DNA, 12 bp, Artificial sequence
  Sequence motif, design or oligomer
gcaagcatcctg

SEQ ID NO: 18 - DNA, 16 bp, Artificial sequence
  Sequence motif, design or oligomer
tggcaagcatcctgta

SEQ ID NO: 19 - DNA, 14 bp, Artificial sequence
  LNA oligomer
ggcaagcatcctgt

SEQ ID NO: 20 - DNA, 12 bp, Artificial sequence
  LNA oligomer
gcaagcatcctg

SEQ ID NO: 21 - DNA, 14 bp, Artificial sequence
  LNA oligomer
ggcaagcatcctgt

SEQ ID NO: 22 - DNA, 14 bp, Artificial sequence
  LNA oligomer
ggcaagcatcctgt

SEQ ID NO: 23 - DNA, 13 bp, Artificial sequence
  LNA oligomer
ggcaagcatcctg

SEQ ID NO: 24 - DNA, 13 bp, Artificial sequence
  LNA oligomer
ggcaagcatcctg

SEQ ID NO: 25 - DNA, 13 bp, Artificial sequence
  LNA oligomer
gcaagcatcctgt

SEQ ID NO: 26 - DNA, 13 bp, Artificial sequence
  LNA oligomer
gcaagcatcctgt

SEQ ID NO: 27 - DNA, 12 bp, Artificial sequence
  LNA oligomer
gcaagcatcctg

SEQ ID NO: 28 - DNA, 16 bp, Artificial sequence
  Motif sequence
tggcaagcatcctgta

SEQ ID NO: 29 - DNA, 16 bp, Artificial sequence
  LNA oligomer
tggcaagcatcctgta

SEQ ID NO: 30 - DNA, 16 bp, Artificial sequence
  LNA oligomer
tggcaagcatcctgta

SEQ ID NO: 31 - DNA, 12 bp, Artificial sequence
  LNA oligomer
gcaagcatcctg

SEQ ID NO: 32 - DNA, 12 bp, Artificial sequence
  LNA oligomer
gcaagcatcctg

SEQ ID NO: 33 - DNA, 12 bp, Artificial sequence
  LNA oligomer
gcaagcatcctg